(19) United States
(12) Patent Application Publication
(10) Pub. No.: US 2008/0107613 A1
(43) Pub. Date: May 8, 2008

(54) Pharmaceutical composition comprising a macrolide t-cell immunomodulator and anti-photoaging agent

(76) Inventors: Thomas Hultsch, Mountain Lakes (US); Carle Paul, Milhouse (FR); Nablia Sekkat, Basel (SE); Stefan Hirsch, Lorrach (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/666,793

(22) PCT Filed: Nov. 14, 2005
(86) PCT No.: PCT/EP05/12177

§ 371 Date: May 1, 2007

(30) Foreign Application Priority Data
Nov. 16, 2004 (GB) 0425255.7

(51) Int. Cl. A61K 31/4745; A61K 38/46; A61K 8/64

(52) U.S. Cl. 424/59; 514/291; 424/70.14; 424/94.6

(57) ABSTRACT

Compositions are provided comprising a macrolide T-cell immunomodulator or immunosuppressant agent, e.g. pimecrolimus, and at least one anti-photoaging agent such as a sunscreen, an anti-oxidant or a DNA-repair enzyme.

They are useful in particular in the curative treatment or prevention of dermatological diseases and of conditions where it is desired to inhibit the skin aging effects of light radiation such as UV-A and UV-B light, e.g. development of skin cancer.

The invention relates to pharmaceutical compositions and their use, in particular in the treatment of skin diseases. It concerns pharmaceutical compositions comprising a macrolide T-cell immunomodulator or immunosuppressant agent and an anti-photoaging agent.

It has now been found that macrolide T-cell immunomodulators and immunosuppressants, when used in combination or association with anti-photoaging agents, such as UV-protecting agents (sunscreens), are surprisingly effective and may even act synergistically, resulting in a potentiation of cosmetic/pharmacological activity, such that a beneficial effect in e.g. photoaging of skin is obtained upon co-administration on combination or association.

Excessive exposure to light radiation, particularly ultraviolet (UV) light, induces premature skin aging and UV induced specific mutations in DNA of keratinocytes. The use of i.a. sunscreens can alleviate this negative effect of UV radiation (K. D. Cooper et al., Exp. Dermatol. 11 [2002], Suppl. 1, 20-27).

Further, an increased incidence of UV-light-related skin tumours is a well-known problem in patients undergoing e.g. post-transplantation immunosuppression with systemic macrolide T-cell immunomodulators or immunosuppressants (calcineurin inhibitors) such as cyclosporin A or tacrolimus, and similar concerns have been voiced as regards exposure to UV light upon topical application. Hence the user information for calcineurin inhibitors usually cautions against excessive sunlight exposure of patients undergoing treatment.

However, it has recently been observed that topically applied calcineurin inhibitors such as tacrolimus and pimecrolimus decrease DNA damage induced by UV light in mouse skin (J. Investig. Dermatol. 121 [No. 1][2003] Abstr. 1072), as measured by the effect of UV-B on thymidine dimer formation, indicative of a photoprotective effect. It was determined thereby that in hairless mice pretreated with 1% pimecrolimus cream, 0.1% tacrolimus ointment or vehicle alone, the amount of epidermal thymine dimers, measured one hour after a dose of 1 J/cm² of UVB irradiation, was decreased by 89%, 84% and 47%, respectively, as compared to untreated mice, and UVB-induced apoptosis was less pronounced in treated mice, suggesting that topical calcineurin inhibitors prevent DNA damage due to a filter effect of both vehicle and active components, and especially of active components.

Similar results are obtained with skin from subjects receiving pimecrolimus, either with or without skin disease (atopic dermatitis), exposed to simulated solar radiation, when DNA is extracted from epidermis biopsied 1 hour and 24 hours after irradiation, as measured by pyrimidine dimer visualization by immunoslot blot and quantification by chemoluminescence image analysis: at 1 hour post-irradiation thymine dimer levels are clearly lower as compared to untreated control, indicative of reduced dimer production in skin sites pretreated with pimecrolimus.

This is in stark contrast to the known fact that UV-mediated skin carcinogenesis is accelerated in the setting of long-term immunosuppression with systemic calcineurin inhibitors, and opens up so far unrecognized potential for pharmacological/cosmetical developments, such as in combined use of prima facie unrelated active agents.

It has now been found that, surprisingly, macrolide T-cell immunomodulators or immunosuppressants are readily compatible with anti-photoaging agents, and may even enhance their beneficial effects, thereby reducing the risk for UV-induced DNA damage and skin inflammation and resulting in overall further enhanced safety of treatment, particularly topical treatment, with calcineurin inhibitor in patients exposed to significant UV radiation, such as a child during summer vacation, and improved convenience of application, such as for caregivers of young children.

The invention concerns pharmaceutical compositions comprising a macrolide T-cell immunomodulator or immunosuppressant agent in combination or association with at least one anti-photoaging agent, together with at least one pharmaceutically and cosmetically acceptable diluent or carrier, hereinafter briefly named “the compositions of the invention”.

A macrolide T-cell immunomodulator or immunosuppressant agent is a T-cell immunomodulator or T-cell immunosuppressant which has a macrocyclic compound structure including a lactone or lactame moiety. While it preferably has at least some T-cell immunomodulating or immunosuppressant activity, it also exhibits concomitantly or predominantly further pharmaceutical properties, especially anti-inflammatory activity. It is for example an FKBP 12-binding calcineurin inhibitor or mitogen-activated kinase modulator or inhibitor, in particular an ascomycin or a rapamycin. It preferably is an ascomycin. It preferably is a compound, e.g. an ascomycin, having rather long-acting activity relatively to other members of the same structural class, e.g. it is metabolically degraded slowly to inactive products.

An asco- or rapamycin is to be understood as asco- or rapamycin as such, or a derivative thereof. A derivative is to be understood as being an antagonist, agonist or analogue of the parent compound which retains the basic structure and modulates at least one of the biological, for example immunological properties of the parent compound.

Suitable ascomycins are e.g. as described in EP 184162, EP 315978, EP 323042, EP 423714, EP 427680, EP 465426, EP 474126, WO 91/13889, WO 91/19495, EP 484936, EP 523088, EP 532089, EP 569337, EP 626385, WO 93/5059 and WO 97/8182; in particular:
ascomycin;
tacrolimus (FK506; Prograf^{R});
imidazolylmethoxyascomycin (WO 97/8182 in Example 1 and as compound of formula I);
32-O-(1-hydroxyethylindol-5-yl)ascomycin (L-732531) (Transplantation 65 [1998] 10-18, 18-26, on page 11, FIG. 1; and
(32-desoxy,32-epi-N1-tetrazolyl)ascomycin (ABT-281) (J. Invest. Dermatol. 12 [1999] 729-738, on page 730, FIG. 1);
   preferably:
{1R,5Z,9S,12S-[1E-(1R,3R,4R)],13R,14S,17R,18E,21S,23S,24R,25S,27R}-17-ethyl-1,14-dihydroxy-12-[2-(4-hydroxy-3-methoxycyclohexyl)-1-methylvinyl]-23,25-dimethoxy-13,19,21,27-tetramethyl-11,28-dioxa-4-azatricyclo[22.3.1.0(4,9)]octacos-5,18-diene-2,3,10,16-tetraone (Example 8 in EP 626385),
hereinafter referred to as “5,6-dehydroascomycin”;
{1E-(1R,3R,4R)]1R,4S,5R,6S,9R,10E,13S,15S,16R,17S,19S,20S}-9-ethyl-6,16,20-trihydroxy-4-[2-(4-hydroxy-3-methoxycyclohexyl)-1-methylvinyl]-15,17-dimethoxy-5,11,13,19-tetramethyl-3-oxa-22-azatricyclo[18.6.1.0(1,22)]heptacos-10-ene-2,8,21,27-tetraone (Examples 6d and 71 in EP 569337), hereinafter referred to as “ASD 732”; and
pimecrolimus (INN recommended) (ASM981; Elidel^{R}), i.e. {[1E-(1R,3R,4S)]1R,9S,12S, 13R,14S,17R,18E,21S,23S,24R,25S,27R}-12-[2-(4-chloro-3-methoxycyclohexyl)-1-methylvinyl]-17-ethyl-1,14-dihydroxy-23,25-dimethoxy-13,19,21,27-tetramethyl-11,28,dioxa-4-azatricyclo [22.3.1.0(4,9)]octacos-18-ene-2,3,10,16-tetraone, of formula I

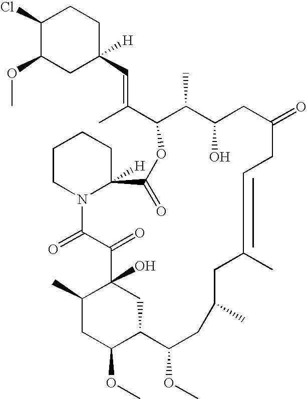

### (Example 66a in EP 427680), also known as “33-epichloro-33-desoxyascomycin”.

Suitable rapamycins are e.g. as described in U.S. Pat. No. 3,929,992, WO 94/9010 and U.S. Pat. No. 5,258,389, preferably sirolimus (rapamycin; Rapamune^{R}) and everolimus (RAD001; Certican^{R}).

Especially preferred as macrolide T-cell immunomodulator or immunosuppressant agent is pimecrolimus, a compound which is particularly well tolerated on skin.

An anti-photoaging agent is a pharmacological or cosmetical agent which protects against the harmful aging effects on skin of UV radiation upon topical application, particularly against actinic keratosis, actinic dermatitis, sunburn, photodermatitis, erythema, UV-induced immunosuppression and their harmful effects related with exposure to light, especially UV light, and photoaging is to be understood herein as including photocarcinogenesis. The anti-photoaging agent is e.g. a topically applied UV-protecting agent, also commonly designated a sunscreen, or an anti-oxidant or anti-oxidant precursor, or topically applied DNA repair enzymes.

Suitable UV-protecting agents (sunscreens) are e.g. UVA and UVB absorbers, preferably compounds absorbing both UVA (315-400 nm) and UVB (280-315 nm), e.g.:
UVB: cinnamates; p-aminobenzoic acid; salicylates; camphor derivatives, such as benzylidene camphor derivatives; benzimidazoles; triazones; etocrylene; octocrylene; and urocanic acid;
UVA: benzophenones; camphor derivatives, such as benzylidene camphor derivatives; dibenzoylmethane derivatives; anthralinates; and bisimidazylate;
UVA and UVB: anisotriazone, drometrizole trisiloxane, methylene bisbenzotriazolyl tetramethylbutylphenol; and inorganic sunscreens such as titanium dioxide and zinc oxide;
e.g. as known under the trademarks Uvinul N-539^{R} (octrylene); Eusolex 232^{R} (phenylbenzimidazole sulfonic acid); Parsol 1789^{R} (avobenzone); Mexoryl SX^{R} (drometizole trisulfonate); Mexoryl XL^{R} (pterethalydene dicamphor sulfonic acid); Tinasorb^{R}, e.g. Tinasorb M^{R} (methylene bisbenzotriazol tetramethylbutylphenol), and Tinasorb S^{R} (anizotriazine);
combinations of sunscreens with broad spectrum protection and a sun protection factor (SPF) of 15 or higher;

They are e.g.:
Coppertone^{R} Shade Sunblock (a mixture of ethylhexyl-p-methoxycinnamate, 2-ethylhexyl salicylate, oxybenzone and homosalate) (Schering-Plough Healthcare Products, Inc.);
PreSun^{R} Ultra Sunscreen (a mixture of octyl methoxycinnamate, octyl salicylate, oxybenzone and avobenzone [Parsol 1789]) (Westwood-Squibb Pharmaceuticals, Inc.);
Ombrelle^{R} Sunscreen (a mixture of octyl methoxycinnamate, oxybenzone and avobenzone [Parsol 1789] (Ombrelle Pharmaceuticals, Inc.); and
Helioblock^{R} (Galderma, La Roche Posay; mixture of e.g. butyl methoxydibenzoylmethane, titanium dioxide and octocrylene).

They may be organic or inorganic.

Further UV-protecting agents (sunscreens) are e.g. the inorganic sunscreens zirconium oxide and iron oxide, and the following organic sunscreens: salts and derivatives of p-aminobenzoic acid such as the ethyl, isobutyl, glyceryl esters, and p-dimethylaminobenzoic acid; anthranilates, e.g. o-amino-benzoates such as the methyl, methyl, phenyl, benzyl, phenylethyl, linalyl, terpenyl and cyclohexenyl esters; further salicylates, e.g. the amyl, phenyl, octyl, benzyl, menthyl, glyceryl and di-propyleneglycol esters; further cinnamic acid derivatives, e.g. the menthyl and benzyl esters, or butylcinnamoyl pyruvate; dihydroxycinnamic acid derivatives such as umbelliferone, methylumbelliferone, methylaceto-umbelliferone; trihydroxycinnamic acid derivatives such as esculetin, methylesculetin, daphnetin and the glucosides esculin and daphnin; hydrocarbons such as diphenylbutadiene and stilbene; dibenzalacetone and benzalacetophenone; naphtholsulfonates, e.g. the sodium salts of 2-naphthol-3,6-disulfonic acid and of 2-naphthol-6,8-disulfonic acid; di-hydroxynaphthoic acid and its salts; o- and p-hydroxybiphenyldisulfonates; coumarin derivatives such as 7-hydroxy, 7-methyl or 3-phenylcoumarin; diazoles, e.g. 2-acetyl-3-bromoindazole, phenyl benzoxazole, methyl naphthoxazole, and aryl benzothiazoles; quinine salts, e.g. the bisulfate, sulfate, chloride, oleate and tannate; quinoline derivatives, e.g. 8-hydroxyquinoline salts and 2-phenylquinoline; hydroxy- or methoxy-substituted benzophenones; uric and violuric acids; tannic acid and its derivatives, e.g. the hexaethylether; (butylcarbotol)(6-propylpiperonyl)ether; hydroquinone; benzophenones, e.g. oxybenzene, sulisobenzone, dioxybenzone, benzoresorcinol, 2,2′,4,4′-tetrahydroxybenzophenone, 2,2′-dihydroxy-4,4′-dimethoxybenzophenone and octabenzone; 4-isopropyldibenzoylmethane; butylmethoxydibenzoylmethane; etocrylene; octocrylene; 3-(4′-methylbenzylidene)bornane-2-one; terephthalylidene dicamphor sulfonic acid; and 4-isopropyl-di-benzoylmethane.

Preferred UV-protecting agents (sunscreens) are p-aminobenzoic acid, titanium dioxide, zinc oxide, pterethalydene dicamphor sulfonic acid (Mexoryl XL^{R}), and micronized organic sunscreens such as methylene bisbenzotriazol tetramethylbutylphenol (Tinasorb M^{R}).

Suitable anti-oxidants or anti-oxidant precursors are e.g.:
polyphenols, e.g. tea polyphenols, e.g. green tea or white tea polyphenol extracts;
isoflavones, such as genistein;
glutathione or a precursor thereof, such as N-acetylcysteine; and
tocopherol and tocopherol derivatives.

Further anti-oxidants or anti-oxidant precursors are e.g.: ascorbic acid (vitamin C) and its salts, ascorbyl esters of fatty acids, ascorbic acid derivatives such as magnesium ascorbyl phosphate, sodium ascorbyl phosphate and ascorbyl sorbate; butylated hydroxy benzoic acids and their salts; 6-hydroxy-2,5,7,8-tetramethylchroman-2-carboxylic acid; gallic acid and its alkyl esters, especially propyl gallate; uric acid and its salts and alkyl esters; sorbic acid and its salts; lipoic acid; amines such as N,N-diethylhydroxylamine and aminoguanidine; sulfhydryl compounds such as glutathione; dihydroxyfumaric acid and its salts; lycine pidolate; arginine pilolate; nordihydroguaiaretic acid; biflavonoids; curcumin; lysine; methionine; proline; superoxide dismutase; silymarin; tea extracts; grape skin/seed extracts; melanin; and rosemary extracts.

Preferred anti-oxidants are tocopherol and tocopherol derivatives, e.g. tocopherol sorbate, especially tocopherol acetate (vitamin E).

While anti-oxidants are exerting their biological function, they may also accessorily confer improved physicochemical stability to the compositions of the invention, e.g. improved stability to oxidation and handling and storage.

Suitable topically applied DNA repair enzymes are e.g. in the form of photolyase-containing liposomes.

Preferred are compositions of the invention comprising several anti-photoaging agents, e.g. at least one UV-protecting agent (sunscreen) and an anti-oxidant. Particularly preferred is pimecrolimus in combination with titanium dioxide and/or pterethalydene dicamphor sulfonic acid (Mexoryl XL^{R}), and/or with tocopherol or tocopherol acetate (vitamin E), especially pimecrolimus in combination with titanium dioxide, or in combination with titanium dioxide, pterethalydene dicamphor sulfonic acid and tocopherol or tocopherol acetate.

The compositions of the invention are adapted for topical administration as regards at least their anti-photoaging agent component.

They may however be adapted for systemic administration as regards their macrolide agent component, e.g. oral or intravenous, or adapted for topical administration of both kinds of component agents; preferably they are adapted for topical administration of both.

They are indicated for use in the known indications of the particular active agents incorporated therein. They are particularly indicated for use in dermatological diseases which have an inflammatory component or involve inflammatory complications, such as erythema post-peel, dry skin, atopic or contact dermatitis, and in photo-protection, e.g. in conditions where it is desired to inhibit the skin aging effects of light radiation, especially of sunlight, e.g. in actinic keratosis, actinic dermatitis, sunburn, photodermatitis, erythema, immunosuppression and their harmful effects related with exposure to light, especially UV light, such as photocarcinogenesis, e.g. development of skin cancer. This is also particularly relevant for patients who have risk factors for or a history of skin cancer.

The composition resulting from the combination or association can be a medicated formulation, appropriately presented, e.g. as a poultice or a cataplasm, or a free combination.

Preferred are compositions comprising an ascomycin in combination with at least one anti-photoaging agent, especially pimecrolimus, preferably in combination with sunscreen or anti-oxidant, or in combination with sunscreen and anti-oxidant.

“Treatment” as used herein refers to use for alleviating an existing condition, i.e. in curative treatment, or prophylactically, i.e. in prevention.

The anti-photoaging agent may be used singly in combination with the macrolide agent, or several such agents may be combined, e.g. a sunscreen and an anti-oxidant.

Some anti-photoaging agents indicated for use in the present invention, such as tocopherol, are known also to be commonly used as e.g. antioxidants in the preparation of pharmaceutical compositions comprising macrolides as active agent. It should be appreciated that in such compositions the antioxidants are included to prevent oxidation in, and thus stabilize, the composition as such, and are present therein in very small amounts, e.g. 0.1% of the total weight of the composition. With the present invention, in contrast thereto, it is contemplated that the amount of anti-photoaging agent normally is considerably larger and can constitute up to about 95% of the total weight, e.g. from about 10% to about 90% w/w.

Synergy is e.g. calculated as described in Berenbaum, Clin. Exp. Immunol. 28 (1977) 1, using an interaction term to correct for differences in mechanism between the two drugs, as described in Chou et al., Transpl. Proc. 26 (1994) 3043. The index of synergy is calculated as:

dose of A/A_{E}+dose of B/B_{E}+(dose of A)×(dose of B)/A_{E}×B_{E}

in which the doses of the compounds A and B represent those used in a particular combination, and A_{E} and B_{E} are the individual doses of A and B respectively giving the same effect. If the result is less than 1, there is synergy; if the result is 1, the effect is additive; if the result is greater than 1, A and B are antagonistic. By plotting an isobologram of dose of A/A_{E} vs. dose of B/B_{E} the combination of maximum synergy can be determined. The synergistic ratio expressed in terms of the ratio by weight of the two compositions at synergistic amounts along the isobologram, especially at or near the point of maximum synergy, can then be used to determine formulations containing an optimally synergistic ratio of the two compounds.

Activity may e.g. be determined in known assay models for testing the activity of the individual components of the compositions.

The molar amount of macrolide agent present is from roughly similar to, to significantly less than the amount of anti-photoaging agent, preferably half as much or less. Synergistic ratios of anti-inflammatory agent to anti-photoaging agent by weight are thus suitably from about 10:1 to about 1:50, preferably from about 5:1 to about 1:20, most preferably from about 1:1 to about 1:15, e.g. about 1:12.

The compositions of the invention can be administered as a free combination, or can be formulated into a fixed combination, which greatly enhances the convenience for the patient.

Absolute dosages of the compounds will vary depending on a number of factors, e.g. the individual compound selected, the route of administration, the desired duration, the rate of release of the active agents and the nature and severity of the condition to be treated. For example, the amount of active agents required and the release rate thereof may be determined on the basis of known in vitro and in vivo techniques, determining how long a particular active agent concentration in the blood plasma remains at an acceptable level for a therapeutic effect.

For example, in prevention and treatment of a dermatological disease as defined above, an initial dosage of about 2-3 times the maintenance dosage is suitably administered, followed by a daily dosage of about 2-3 times the maintenance dosage for a period of from one to two weeks, and subsequently the dose is gradually tapered down at a rate of about 5% per week to reach the maintenance dosage. Thus synergistically effective amounts of, in particular, pimecrolimus and sunscreen on oral administration as regards pimecrolimus and topical administration as regards sunscreen, for use in treatment of dermatological diseases as defined above in larger animals, e.g. man, are amounts of pimecrolimus of up to about 2 mg/kg/day, e.g. from about 0.01 mg/kg/day to about 2 mg/kg/day, preferably about 0.5 mg/kg/day, in combination or co-administration with conventional amounts of sunscreen in topical use of up to about 50 mg/kg/day, e.g. from about 0.25 mg/kg/day to about 50 mg/kg/day, preferably about 2.5 mg/kg/day, in a synergistic ratio, as described. Suitable unit dosage forms for oral administration of the macrolide agent thus may contain on the order of from about 0.5 mg to about 100 mg, preferably about 3 mg to about 30 mg of agent, e.g. pimecrolimus. The daily dosage for oral administration is preferably taken in a single dose, but may be spread out over two, three or four dosages per day. For i.v. administration, the effective dosage is lower than that required for oral administration, e.g. about one fifth the oral dosage. For topical administration of the macrolide agent the amount of that agent is e.g. up to about 10 mg/kg/day, e.g. for pimecrolimus from about 0.025 mg/kg/day to about 5 mg/kg/day, e.g. at a 1-3% concentration.

By “co-administration” is meant administration of the components of the compositions of the invention together or at substantially the same time, e.g. within fifteen minutes or less, either in the same vehicle or in separate vehicles, so that upon topical administration, for example, both compounds are present simultaneously on the skin. Preferably, the compounds are administered as a fixed combination for topical use.

The compositions of the invention include fixed compositions suitable for administration by any conventional topical route, in particular in the form of a dermal cream, spray, ointment, mousse, shampoo, solution, lotion, gel, emulgel or like preparation, especially as a cream; they have e.g. a concentration of from about 0.1% to about 3%, preferably about 1% by weight of each kind of component.

The compositions of the invention can be prepared in conventional manner, e.g. by mixing a macrolide T-cell immunomodulator or immunosuppressant agent with at least one anti-photoaging agent, and at least one pharmaceutically and cosmetically acceptable diluent or carrier, e.g. for cream preparation, according to the conventional manufacturing procedure for an emulsion.

The active agent components may be in free form or pharmaceutically acceptable salt form where such forms exist.

The invention also provides products and methods for co-administration of a macrolide T-cell immunomodulator or immunosuppressant agent, e.g. pimecrolimus, tacrolimus or 5,6-dehydroascomycin, and an anti-photoaging agent, e.g. a sunscreen and/or an anti-oxidant, at pharmaceutically effective dosages, e.g.:
a method of curative treatment or prevention of a dermatological disease which has an inflammatory component or involves inflammatory complications, or of conditions where it is desired to inhibit the skin aging effects of light radiation, in a subject suffering from or at risk for such condition, comprising co-administering a pharmaceutically and cosmetically effective amount of a composition of the invention;
the use of a macrolide T-cell immunomodulator or immunosuppressant agent in the manufacture of a medicament for co-administration in pharmaceutically effective amounts with at least one anti-photoaging agent;
the use of an anti-photoaging agent in the manufacture of a medicament for co-administration in pharmaceutically effective amounts with a macrolide T-cell immunomodulator or immunosuppressant agent;
a kit of parts comprising a macrolide T-cell immunomodulator or immunosuppressant agent and an anti-photoaging agent in separate unit dosage forms, preferably wherein the unit dosage forms are suitable for administration of the component agents in pharmaceutically and cosmetically effective amounts, together with instruction for use, optionally with further means for facilitating compliance with the administration of the component compounds, e.g. a label or drawings;
the use of a macrolide T-cell immunomodulator or immunosuppressant agent in the manufacture of a pharmaceutical kit which is to be used for facilitating co-administration with at least one anti-photoaging agent;
the use of an anti-photoaging agent in the manufacture of a pharmaceutical kit which is to be used for facilitating co-administration with a macrolide T-cell immunomodulator or immunosuppressant agent; and
a macrolide T-cell immunomodulator or immunosuppressant agent and an anti-photoaging agent, as a combined pharmaceutical preparation for simultaneous, separate or sequential use, preferably in synergistically effective amounts, e.g. for the curative treatment or prevention of a dermatological disease as defined above or of conditions where it is desired to inhibit the skin aging effects of light radiation.

The following Examples illustrate the invention. The compounds are in free, i.e. neutral or base form unless specified otherwise.

### EXAMPLE 1

### Cream (Occlusive) with Mineral Sunscreen

| Component | Amount (g) |
|---|---|
| Pimecrolimus | 1.00 |
| Oily phase: | |
| titanium dioxyde (sunscreen) | 7.50 |
| triglycerides, medium chain | 15.00 |
| oleyl alcohol | 10.00 |
| sodium cetylstearyl sulfate | 1.00 |
| cetyl alcohol | 4.00 |
| stearyl alcohol | 4.00 |
| glyceryl monostearate | 2.00 |
| Water phase: | |
| benzyl alcohol | 1.00 |
| propylene glycol | 5.00 |
| citric acid | 0.05 |
| sodium hydroxide | * |
| water | ad 100.0 |

* as required to adjust pH to 5.5

Preparation is conventional: drug substance is added to the heated homogeneous oily phase. In parallel, the water phase is heated at the same temperature as the oily phase. The oily phase is added to the water phase, homogenization is performed and the resultant cream is cooled to room temperature.

### EXAMPLE 2

### Cream (Occlusive) with a Combination of Sunscreens and Anti-Oxidant

| Component | Amount (g) |
|---|---|
| Pimecrolimus | 1.00 |
| Oily phase: | |
| pterethalydene dicamphor sulfonic acid | 8.00 |
| (Mexoryl XL^{R}) (sunscreen) | |
| titanium dioxide (sunscreen) | 1.00 |
| triglycerides, medium chain | 15.00 |
| oleyl alcohol | 10.00 |
| sodium cetylstearyl sulfate | 1.00 |
| cetyl alcohol | 4.00 |
| stearyl alcohol | 4.00 |
| glyceryl monostearate | 2.00 |
| tocopheryl acetate (vitamin E) | 0.45 |
| (anti-oxidant) | |
| Water phase: | |
| benzyl alcohol | 1.00 |
| propylene glycol | 5.00 |
| citric acid | 0.05 |
| sodium hydroxide | * |
| water | ad 100.0 |

* as required to adjust pH to 5.5

Preparation is conventional and effected as described for Example 1 above.

1. A pharmaceutical composition comprising a macrolide T-cell immunomodulator or immunosuppressant agent in combination or association with at least one anti-photoaging agent, together with at least one pharmaceutically and cosmetically acceptable diluent or carrier,
whereby the macrolide is other than tacrolimus or rapamycin when an anti-oxidant is sole anti-photoaging agent and is tocopherol, butylhydroxyanisol, butylhydroxytoluene, ascorbic acid or a salt thereof or an ascorbyl ester of a fatty acid.

2. A composition according to claim 1 wherein the macrolide T-cell immunomodulator or immunosuppressant agent is pimecrolimus or tacrolimus.

3. A composition according to claim 1 wherein the macrolide T-cell immunomodulator or immunosuppressant agent is pimecrolimus.

4. A composition according to claim 1 wherein the anti-photoaging agent is a UV-protecting agent (sunscreen).

5. A composition according to claim 1 wherein the anti-photoaging agent is an anti-oxidant.

6. A composition according to claim 1 wherein the anti-photoaging agent is a DNA-repair enzyme.

7. A composition according to claim 1 wherein the anti-photoaging agent comprises titanium dioxide, or a mixture of titanium dioxide, pterethalydene dicamphor sulfonic acid, and tocopherol or tocopherol acetate.

8. A method of curative treatment or prevention of a dermatological disease which has an inflammatory component or involves inflammatory complications, or of conditions where it is desired to inhibit the skin aging effects of light radiation, in a subject suffering from or at risk for such condition, comprising co-administering a pharmaceutically and cosmetically effective amount of a composition according to claim 1.

9. A kit of parts comprising a macrolide T-cell immunomodulator or immunosuppressant agent and an anti-photoaging agent according to claim 1 in separate unit dosage forms, together with instructions for use.

10. A process for the preparation of a composition according to claim 1 comprising mixing a macrolide T-cell immunomodulator or immunosuppressant agent with at least one anti-photoaging agent, and at least one pharmaceutically and cosmetically acceptable diluent or carrier.
